SEQUENCE LISTING (39 sequences)

SEQ ID NO: 1 - protein, 6 aa, Artificial Sequence
  Synthetic
XXXXXX

SEQ ID NO: 2 - protein, 6 aa, Artificial Sequence
  Synthetic
RRGHYY

SEQ ID NO: 3 - protein, 6 aa, Artificial Sequence
  Synthetic
RRXHYY

SEQ ID NO: 4 - protein, 6 aa, Artificial Sequence
  Synthetic
RRGXYY

SEQ ID NO: 5 - protein, 6 aa, Artificial Sequence
  Synthetic
RRGHXY

SEQ ID NO: 6 - protein, 6 aa, Artificial Sequence
  Synthetic
RRGHYX

SEQ ID NO: 7 - protein, 13 aa, Artificial Sequence
  Synthetic
HSAYSDTRRGDYG

SEQ ID NO: 8 - protein, 6 aa, Artificial Sequence
  Synthetic
RRGDYG

SEQ ID NO: 9 - protein, 6 aa, Artificial Sequence
  Synthetic
RRGDYH

SEQ ID NO: 10 - protein, 6 aa, Artificial Sequence
  Synthetic
RRGHYG

SEQ ID NO: 11 - protein, 13 aa, Artificial Sequence
  Synthetic
HSACSDTRRGDCG

SEQ ID NO: 12 - DNA, 20 bp, Artificial Sequence
  Synthetic
ccctcatagttagcgtaacg

SEQ ID NO: 13 - protein, 6 aa, Artificial Sequence
  Synthetic
AAGHYY

SEQ ID NO: 14 - protein, 7 aa, Artificial Sequence
  Synthetic
LRRASLG

SEQ ID NO: 15 - protein, 7 aa, Artificial Sequence
  Synthetic
RRKASGP

SEQ ID NO: 16 - protein, 9 aa, Artificial Sequence
  Synthetic
CSDTRRGDC

SEQ ID NO: 17 - protein, 9 aa, Artificial Sequence
  Synthetic
CKLGPIRGC

SEQ ID NO: 18 - protein, 9 aa, Artificial Sequence
  Synthetic
CLTSHNMMC

SEQ ID NO: 19 - protein, 2 aa, Artificial Sequence
  Synthetic
RR

SEQ ID NO: 20 - protein, 7 aa, Artificial Sequence
  Synthetic
LRRASLG

SEQ ID NO: 21 - protein, 7 aa, Artificial Sequence
  Synthetic
RRKASGP

SEQ ID NO: 22 - DNA, 21 bp, Artificial Sequence
  Synthetic
agtgatactcggaggggtgat

SEQ ID NO: 23 - protein, 7 aa, Artificial Sequence
  Synthetic
SDTRRGD

SEQ ID NO: 24 - DNA, 21 bp, Artificial Sequence
  Synthetic
aagcttggtccgattcggggt

SEQ ID NO: 25 - protein, 7 aa, Artificial Sequence
  Synthetic
KLGPIRG

SEQ ID NO: 26 - DNA, 21 bp, Artificial Sequence
  Synthetic
ctgacgtctcataatatgatg

SEQ ID NO: 27 - protein, 7 aa, Artificial Sequence
  Synthetic
PWTKAYH

SEQ ID NO: 28 - DNA, 21 bp, Artificial Sequence
  Synthetic
ccttggacgaaggcttatcat

SEQ ID NO: 29 - protein, 7 aa, Artificial Sequence
  Synthetic
NSYSLSR

SEQ ID NO: 30 - DNA, 21 bp, Artificial Sequence
  Synthetic
aattcttattcgctgtctcgg

SEQ ID NO: 31 - protein, 7 aa, Artificial Sequence
  Synthetic
RSHTPRS

SEQ ID NO: 32 - DNA, 21 bp, Artificial Sequence
  Synthetic
cgctctcatactccgcgtagt

SEQ ID NO: 33 - protein, 7 aa, Artificial Sequence
  Synthetic
RSHTPRS

SEQ ID NO: 34 - protein, 286 aa, Serratia marcescens [sequence omitted]

SEQ ID NO: 35 - protein, 286 aa, Pseudomonas aeruginosa [sequence omitted]

SEQ ID NO: 36 - protein, 286 aa, Klebsiella pneumoniae [sequence omitted]

SEQ ID NO: 37 - protein, 126 aa, Staphylococcus aureus
MANKQVEISMAEWDVMNIIWGKKSVSANEIVVEIQKYKEVSDKTIRTLITRLYKKEIIKRYKSENIYFYSSNIKEDDIKMKTAKTFLNKLYGGDMKSLVLNFAKNEELNNKEIEELRDILNDISKK

SEQ ID NO: 38 - protein, 309 aa, Bacillus anthracis
MIVLKNKKMLKIGMCVGILGLSITSLVTFTGGALQVEAKEKTGQVKHKNQATHKEFSQLEKKFDARLGVYAIDTGTNQTIAYRPNERFAFASTYKALAAGVLLQQNSTKKLDEVITYTKEDLVDYSPVTEKHVDTGMTLGEIAEAAVRYSDNTAGNILFHKIGGPKGYEKALRQMGDRVTMSDRFETELNEAIPGDIRDTSTAKAIATNLKAFTAGNALPNHKRNILTKWMKGNATGDKLIRAGVPTNWVVADKSGAGSYGTRNDIAIVWPPNRAPIIIAILSSKDEKGATYDNQLIAEAAEVIVNAFR

SEQ ID NO: 39 - protein, 381 aa, Enterobacter cloacae
MMRKSLCCALLLGISCSALATPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVEGSDSKVALAPLPVAEVNPPAPPVKASWVHKTGSTGGFGSYVAFIPEKQIGIVMLANTSYPNPARVEAAYHILEALQ

(19) United States
(12) Patent Application Publication
(10) Pub. No.: US 2005/0186197 A1
(43) Pub. Date: Aug. 25, 2005

(54) Peptide inhibitors of beta lactamases

(76) Inventors: Timothy Palzkill, Houston, TX (US); Wanzhi Huang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/059,226

(22) Filed: Feb. 16, 2005

Related U.S. Application Data
(63) Continuation of application No. PCT/US03/27275, filed on Aug. 29, 2003.
(60) Provisional application No. 60/406,806, filed on Aug. 29, 2002.

(51) Int. Cl. A61K 38/54; C12N 9/99

(52) U.S. Cl. 424/94.3; 435/184

(57) ABSTRACT

Peptide inhibitors of β-lactamases have been identified by the synthesis of peptide arrays using synthesis SPOT technology. These peptide inhibitors of β-lactamase have activity against a broad spectrum of β-lactamases and are useful in a variety of applications.

### CROSS-REFERENCE TO RELATED APPLICATIONS

This application is a continuation of International Application No. PCT/US03/27275, filed Aug. 29, 2003, which claims priority to U.S. Provisional Application No. 60/406,806, filed Aug. 29, 2002, and both applications are hereby incorporated by reference in their entireties herein.

### STATEMENT REGARDING FEDERALLY SPONSORED RESEARCH OR DEVELOPMENT

This invention was developed with funds from the National Institutes of Health, under grant number A132956. The U.S. government may have certain rights in this invention.

### TECHNICAL FIELD

The field of the invention relates to inhibitors of β-lactamases. More particularly, the invention relates to peptide inhibitors of β-lactamases.

### BACKGROUND OF THE INVENTION

The increased resistance of bacterial pathogens to clinically useful antibiotics has become a serious public health threat. It is therefore critical to identify new antimicrobials or to design inhibitors of antibiotic resistance conferring enzymes. The β-lactam antibiotics such as the penicillins and cephalosporins are among the most often used antimicrobial agents. As with other antibiotics, resistance to these agents has been increasing in recent years. The most common mechanism of bacterial resistance to β-lactam antibiotics is the production of β-lactamases (Livermore, 1995). These enzymes are secreted by both gram-positive and gram-negative bacteria and provide resistance by catalyzing the hydrolysis of the β-lactam ring that is common to all antibiotics of this class.

β-lactamases have been grouped into four classes based on primary sequence homology. Classes A, C and D are active-site serine enzymes that catalyze the hydrolysis of the β-lactam via a serine-bound acyl intermediate (Ghuysen, 1991). Class B enzymes require zinc for activity and catalysis does not proceed via a covalent intermediate (Bush, 1998; Carfi et al., 1995; Wang et al., 1999). The active-site serine β-lactamases belong to a larger family of penicillin-recognizing enzymes that includes the penicillin binding proteins (PBPs) that crosslink bacterial cell walls (Massova and Mobashery, 1998). The most prevalent plasmid encoded β-lactamase in Gram-negative bacteria is the class A TEM-1 β-lactamase that catalyzes the hydrolysis of both penicillins and cephalosporins (Frere et al., 1999). Extended-spectrum cephalosporins have been introduced in an effort to circumvent the action of TEM-1 β-lactamase. The use of these agents, however, has resulted in the emergence of TEM mutant derivatives capable of hydrolyzing extended spectrum antibiotics (Petrosino et al., 1998).

An alternative method to combat β-lactamase mediated resistance has been the use of mechanism-based, small molecule inhibitors such as clavulanic acid and sulbactam (Bush, 2002). These inhibitors protect the β-lactam drug from hydrolysis by β-lactamases and restore the therapeutic potential of the antibiotic (Bush, 2002; Charnas and Knowles, 1981). Variants have now evolved, however, that resist these inhibitors while maintaining the ability to hydrolyze β-lactam antibiotics (Henquell et al., 1995; Imtiaz et al., 1994; Petrosino et al., 1998). Therefore, a need exists for the development of new inhibitors. Phage display is a powerful technique for studying protein-ligand interactions (reviewed by (Smith and Petrenko, 1997)). The method involves the fusion of peptides or proteins to a coat protein of a filamentous bacteriophage (Smith, 1985). The peptides or proteins are normally fused to the N-terminus of the gene III phage protein. The gene III protein is a minor coat (3-5 copies per phage) protein located at the tip of the phage and is responsible for attachment of the phage to the bacterial F pilus in the course of the normal infection process (Rasched and Oberer, 1986). Because the gene encoding the fusion protein is packaged within the same phage particle, there is a direct link between the phenotype, i.e., the ligand binding characteristics of a displayed peptide, and the DNA sequence of the gene for the displayed peptide. This permits large libraries of peptides of random amino acid sequence to be rapidly screened for desired ligand binding properties (Smith and Petrenko, 1997).

Although phage display can be used to identify peptide ligands, these ligands generally do not bind the target protein with high affinity except in cases where the protein normally functions in peptide recognition (Clackson and Wells, 1994; Cochran, 2001). Peptide arrays offer a rapid means of optimizing the binding properties of peptides identified using phage display (Reimer et al., 2002; Reineke et al., 2001). The SPOT synthesis method, for example, can be used to create large arrays of synthetic peptides on cellulose filters (Frank, 1992). The method employs Fmoc protection chemistry whereby the reagents are delivered automatically to discrete spots on the filters (Reineke et al., 2001). The resulting array can be screened directly in the solid phase using an appropriately labeled target protein to identify peptides that bind the target with increased affinity (Reimer et al., 2002).

A combination of phage display and SPOT synthesis were used here to identify and optimize peptides that bind and inhibit TEM-1 β-lactamase. Surprisingly, the peptides optimized for binding the TEM-1 enzyme also inhibited the class A Bacillus anthracis Bla1 enzyme and the class C β-lactamase, P99. These broad-spectrum peptide inhibitors may serve as the basis for the design of peptidomimetics that inhibit a wide range of β-lactamases.

### BRIEF SUMMARY OF THE INVENTION

An embodiment of the invention is a peptide inhibitor of β-lactamase comprising X′₁′₂X₁X₂X₃X₄ (SEQ ID NO:1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

In a specific embodiment, the peptide inhibitor of β-lactamase comprises RRGHYY (SEQ ID NO:2).

In another specific embodiment, the peptide inhibitor of β-lactamase comprises a peptide selected from the group consisting of RRX₁HYY (SEQ ID NO:3), RRGX₂YY (SEQ ID NO:4), RRGHX₃Y (SEQ ID NO:5), and RRGHYX₄ (SEQ ID NO:6).

In another specific embodiment, the peptide inhibitor of β-lactamase comprises a peptide selected from the group consisting of HSAYSDTRRGDYG (SEQ ID NO:7), RRGDYG (SEQ ID NO:8), RRGDYH (SEQ ID NO:9), and RRGHYG (SEQ ID NO:10).

In a specific embodiment of the invention, the β-lactamase is a class A, or C β-lactamase. In a further specific embodiment, the β-lactamase is TEM-1, Bla1, or P99.

In yet another specific embodiment of the invention, the inhibition constant (Kᵢ) is less than about 100 μm.

An embodiment of the invention is a pharmaceutical composition comprising a therapeutically effective amount of a peptide inhibitor of β-lactamase as described. A specific embodiment is a pharmaceutical composition comprising a therapeutically effective amount of a β-lactam antibiotic.

In another specific embodiment, the β-lactam antibiotic is selected from the group consisting of penicillin, cephalosporin, monobactam and carbapenem antibiotics. In yet another specific embodiment, the antibiotic is penicillin.

In a specific embodiment of the invention, the penicillin is selected from the group consisting of azlocillin, methicillin, nafcillin, cloxacillin, dicloxacillin, oxacillin, ampicillin, bacampicillin, carbenicillin, ticarcillin, mezlocillin, penicillin, and piperacillin.

In another specific embodiment, the antibiotic is a cephalosporin. In a further specific embodiment, the cephalosporin is selected from the group consisting of cefoxitin, cefoperazone, ceftazidime, ceftriaxone, cefadroxil, cefazolin, cephalexin, cephaloridine, cephalothin, cephapirin, cephradine, cefaclor, cefamandole, cefonicid, ceforanide, cefprozil, cefuroxime, loracarbef, cefmetazole, cefotetan, cefixime, cefotaxime, cefpodoxime, and ceftizoxime.

An embodiment of the invention is a DNA expression vector comprising a promoter operatively linked to a nucleotide sequence wherein the nucleotide sequence encodes a peptide inhibitor of β-lactamase comprising X′₁X′₂X₁X₂X₃X₄ (SEQ ID NO: 1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

An embodiment of the invention is a host cell capable of expressing a peptide inhibitor of β-lactamase comprising X′₁X′₂X₁X₂X₃X₄ (SEQ ID NO: 1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

An embodiment of the invention is a method of inhibiting a β-lactamase comprising contacting the β-lactamase with a peptide inhibitor of β-lactamase, wherein the peptide inhibitor of β-lactamase comprises X′₁X′₂X₁X₂X₃X₄ (SEQ ID NO: 1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

An embodiment of the invention is a method of inhibiting the growth of a microorganism comprising contacting the microorganism with a β-lactam antibiotic and a peptide inhibitor of β-lactamase, wherein the peptide inhibitor of β-lactamase comprises X′₁X′₂X₁X₂X₃X₄ (SEQ ID NO: 1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

In a specific embodiment, the microorganism is selected from the group consisting of Staphylococcus aureus, Staphylococcus epidermidis and other coagulase-negative staphylococci, Streptococcus pyogenes, Streptococcus pneumoniae, Streptococcus agalactiae, Enterococcus species, Corynebacterium dipahtheriae, Listeria monocytogenes, Bacillus anthracis, Neisseria meningitidis, Neisseria gonorrhoeae, Moraxella catarrhalis, Vibrio cholerae, Campylobacter jejuni, Enterobacteriaceae (includes: Escherichia, Salmonella, Klebsiella, Enterobacter), Pseudomonas aeruginosa, Acinetobacter species, Haemophilus influenzae, Clostridium tetani, Clostridium botulinum, Bacteroides species, Prevotella species, Porphyromonas species, Fusobacterium species, Mycobacterium tuberculosis, and Mycobacterium leprae.

In another specific embodiment, the microorganism is resistant to one or more β-lactam antibiotics.

An embodiment of the invention is a method of treating a subject infected with a microorganism comprising administering to the subject a therapeutically effective amount of a β-lactam antibiotic and a peptide inhibitor of β-lactamase, whererein the peptide inhibitor of β-lactamase comprises X′₁X′₂X₁X₂X₃X₄ (SEQ ID NO:1); wherein X′₁ and X′₂ are arginine or lysine and are the same or different; wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; wherein any of X₁, X₂, X₃, and X₄ are the same or different; and wherein the peptide inhibitor of β-lactamase has an inhibition constant (Kᵢ) for the β-lactamase of no greater than about 500 μm.

### BRIEF DESCRIPTION OF THE DRAWINGS

FIG. 1. Results of DNA sequencing of selected phage clones. Shown are the DNA and protein sequences of peptides selected after three rounds of biopanning on immobilized TEM-1 β-lactamase. The fractions to the right of the sequences indicate the number of times a particular sequence was identified out of the total of 23 clones sequenced.

FIG. 2. Phage ELISA measurement of binding of selected phage to immobilized TEM-1 β-lactamase. Black bars indicate binding of phage to TEM-1 β-lactamase. Gray bars indicate binding of phage to E. coli maltose binding protein.

FIG. 3. Results of TEM-1 β-lactamase binding to peptides containing all single amino acid substitutions in the starting peptide HSACSDTRRGDCG (SEQ ID NO: 11). Each row represents the 20 amino acid substitutions at the listed position of the peptide. Note that the amino acid positions flanking the cysteines (HSACSDTRRGDCG (SEQ ID NO: 11)) were not substituted but are present in each substituted peptide on the array. Each column displays the indicated amino acid substitution at a particular position in the peptide.

FIG. 4. Results of TEM-1 β-lactamase binding to peptides containing all single amino acid substitutions in the starting peptide RRGHYG (SEQ ID NO: 10). Each row represents the 20 amino acid substitutions at the listed position of the peptide. Each column displays the indicated amino acid substitution at a particular position in the peptide.

FIG. 5. Inhibition patterns of TEM-1, Bla1 and P99 β-lactamases by the RRGHYYNH2 (SEQ ID NO:2) peptide. A. Inhibition of TEM-1 β-lactamase evaluated by determining the Kₘ and Vₘₐₓ for cephalosporin C hydrolysis with (control) 0 μM (circles), 50 μM (squares), and 100 μm (triangles) RRGHYY—NH₂. Triangles, 100 μM RRGHYY—NH₂ Kₘ=1179±50 μm; Vₘₐₓ=118±12 μM/min. Squares, 50 μm RRGHYY—NH₂ Kₘ=771±31 μM; Vₘₐₓ=105±5 μm/min. Circles, (control) 0 μM RRGHYY—NH₂Kₘ=598±27 μM; Vₘₐₓ=103±6 μM/min. B. Inhibition of Bla1 β-lactamase evaluated by determining the Kₘ and Vₘₐₓ for PenV hydrolysis with 0 μM (circles), 15 μm (squares), and 30 μm (triangles) RRGHYY—NH₂. Triangles, 30 μm RRGHYY—NH₂ Kₘ=609±14 μM; Vₘₐₓ=243±21 μm/min. Squares, 15 μm RRGHYY—NH₂ Kₘ=450±23 μm; Vₘₐₓ=250±11 μm/min. Circles, (control) 0 μM RRGHYY—NH₂ Kₘ=130±19 μm; Vₘₐₓ=256±16 μm/min. C. Inhibition of P99 β-lactamase evaluated by determining the Kₘ and Vₘₐₓ for cephalosporin C hydrolysis with (control) 0 μM (circles), 50 μM (squares), and 100 μm (triangles) RRGHYY—NH₂. Triangles, 100 μm RRGHYY—NH₂ Kₘ=1387±88 μM; Vₘₐₓ=630±47 μM/min. Squares, 50 μM RRGHYY—NH₂ Kₘ=981±71 μM; Vₘₐₓ=590±64 μM/min. Circles, (control) 0 μm RRGHYYNH₂ Kₘ=774±42 μM; Vₘₐₓ=674±54 μm/min.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, an “amino acid” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

As used herein, the term “β-lactam antibiotic refers to antibiotics containing a β-lactam ring structure. Examples of β-lactam antibiotics are antibiotics from the penicillin or cephalosporin group of antibiotics. The penicillin group of antibiotics includes penicillin G, penicillin V, ampicillin, amoxicillin, hetacillin, methicillin, cloxacillin, dicloxacillin, nafcillin, oxacillin, azlocillin, carbenicillin, mezlocillin, piperacillin, and ticarcillin. The cephalosporin group of antibiotics includes cephalothin, cephalexin, cephapirin, cefadroxil, cephradine, cefazolin, cefaclor, cefamandole, cefrnetazole, cefonicid, ceforanide, cefotetan, cefoxitin, cefprozil, cefuroxime, loracarbef, ceftiofur, cefixime, cefoperazone, cefotaxime, cefpodoxime, ceftiazidime, ceftizoxime, ceftriaxone, and moxalactam.

As used herein, the term “β-lactamase” denotes a protein capable of inactivation of a β-lactam antibiotic. In one preferred embodiment, the β-lactamase is an enzyme which catalyzes the hydrolysis of the β-lactam ring of a β-lactam antibiotic. In certain preferred embodiments, the β-lactamase is microbial. In certain preferred embodiments, the β-lactamase is a serine β-lactamase. In certain other preferred embodiments, the β-lactamase is a zinc β-lactamase. The terms “class A”, “class B”, “class C”, and “class D” β-lactamases are understood by those skilled in the art and can be found described in Waley, The Chemistry of β-Lactamase, Page Ed., Chapman & Hall, London, (1992) 198-228.

As used herein, a “β-lactamase inhibitor” causes a decrease in the activity of β-lactamase when said inhibitor is contacted with the beta-lactamase. This decrease in activity can be measured by a decrease in the ability to cleave a β-lactam antibiotic, such as cephalosporin. It is contemplated that the decrease in cleaving activity may be measured spectrophotometrically. A β-lactamase inhibitor may enhance the activity of a β-lactam antibiotic when administered in combination with said antibiotic. A “peptide inhibitor of β-lactamase” comprises a peptide that, when contacted with the β-lactamase effects a decrease in the ability of the β-lactamase to cleave a β-lactam antibiotic. It is contemplated that a peptide inhibitor of β-lactamase may have inhibitory activity against a broad spectrum of β-lactamases. It is contemplated that the peptide inhibitor of β-lactamase may be a short peptide, and may be 4-20 amino acids in length. It is contemplated that the peptide inhibitor may contain a carboxy-terminal —NH₂ group. It is contemplated that the peptide inhibitor of β-lactamase may be part of a larger polypeptide, and may be either N-terminal, C-terminal, or embedded in the sequence of the larger polypeptide. The larger polypeptide may cause increased solubility of the peptide inhibitor of β-lactamase. The larger polypeptide may also be involved in targeting and delivery of the peptide of inhibitor of β-lactamase to the appropriate location.

A “β-lactam resistant microorganism” is a microorganism with the ability to synthesize a protein that neutralizes or cleaves a β-lactam antibiotic.

The term “bacteriophage” or “phage” as used herein is defined as a virus that infects bacteria. Phages, like other viruses, can be divided into those with RNA genomes e.g., mostly small and single stranded, those with small DNA genomes, e.g., generally less than 10 kb, mostly single stranded, and those with medium to large DNA genomes, e.g., 30-200 kb.

The term “cell wall” as used herein is defined as the peptidoglycan structure of eubacteria which gives shape and rigidity to the cell.

A used herein, “enhance the activity of an antibiotic” or “enhance the activity of a β-lactam antibiotic” refers to the ability of a peptide inhibitor of β-lactamase to increase the ability of an antibiotic to cause inhibition of the growth of a microorganism. The microorganism may or may not be resistant to the antibiotic.

The term “envelope” as used herein is defined as the covering of bacteria which includes the cell wall, its connections to the outer membrane in Gram-negative bacteria, the outer membrane itself, including the lipopolysaccharide, and other outer components such flagella, pili, capsule and other proteins, such as M protein or S-layer proteins.

The term “Gram-negative bacteria” or “Gram-negative bacterium” as used herein is defined as bacteria which have been classified by the Gram stain as having a red stain. Gram-negative bacteria have thin walled cell membranes consisting of a single layer of peptidoglycan and an outer layer of lipopolysaccharide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary organisms not in the family Enterobacteriacea include, but are not limited to, Pseudomonas aeruginosa, Stenotrophomonas maltophilia, Burkholderia, Cepacia, Gardenerella, Vaginalis, and Acientobacter species.

The term “Gram-positive bacteria” or “Gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

As used herein, “inhibition constant”, or “Kᵢ”, refers to the equilibrium constant for the release (or dissociation) of the peptide inhibitor of β-lactamase from the β-lactamase. Smaller numbers, expressed as concentration, indicate better inhibition. One with skill in the art is aware of methods of calculating Kᵢ according to well known methods in the art. It is contemplated that in certain embodiments, the peptide inhibitors of β-lactamase will inhibit β-lactamase with an inhibition constant of no greater than 500 μm.

As used herein, “inhibiting the growth” of a microorganism means reducing by contact with an agent, the rate of proliferation of such a microorganism, in comparison with a control microorganism of the same species not contacted with this agent.

The term “polypeptide” as used herein is defined as a chain of amino acid residues, usually having a defined sequence. As used herein the term polypeptide is mutually inclusive of the terms “peptides” and “proteins”.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

A “subject” is a plant or a vertebrate such as a fish, an avian or a mammal, and preferably a human. Fish include, but are not limited to pets and farm animals. Avians include, but are not limited to pets, sport animals and farm animals. Mammals include, but are not limited to, murines, simians, humans, farm animals, sport animals, and pets.

I. Nucleic Acids

As discussed below, a “nucleic acid sequence” may contain a variety of different bases and yet still produce a corresponding polypeptide that is functionally indistinguishable.

Similarly, any reference to a nucleic acid may be read as encompassing a host cell containing that nucleic acid and, in some cases, capable of expressing the product of that nucleic acid.

A. Nucleic Acids Encoding a Target Polypeptide or Lysis Polypeptide

Nucleic acids according to the present invention may encode a β-lactamase inhibitor peptide as set forth herein.

The term “cDNA” is intended to refer to DNA prepared using messenger RNA (mRNA) as a template. Many of the viruses contain a RNA genome. It is contemplated to utilize these RNA genomes to screen for lysis polypeptides, thus, the RNA would be converted into DNA by standard methods of making “cDNA” from RNA.

It also is contemplated that a given β-lactamase inhibitor peptide may be represented by natural variants that have slightly different nucleic acid sequences but, nonetheless, encode the same protein (see Table 1 below).

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of known sequences for bacterial target proteins and or lysis proteins are contemplated.

The DNA segments of the present invention include those encoding biologically functional equivalent β-lactamase inhibitor peptides, as described above. Such sequences may arise as a consequence of codon redundancy and amino acid finctional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Vectors for Cloning, Gene Transfer and Expression

Within certain embodiments expression vectors are employed to express a β-lactamase inhibitor peptide. Furthermore, it is within the scope of the present invention that the expression vectors may be used. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing cell clones expressing the products are also provided, as is an element that links expression of the drug selection markers to expression of the polypeptide.

(i) Regulatory Elements

Throughout this application, the term “expression construct” or “expression cassette” is meant to include any type of genetic construct containing a nucleic acid coding for a gene product in which part or all of the nucleic acid encoding sequence is capable of being transcribed. In certain embodiments, expression includes both transcription and translation of a β-lactamase inhibitor.

In certain embodiments, the nucleic acid encoding a β-lactamase inhibitor peptide is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box.

In the bacterial genome, there are several conserved features in a bacterial promoter: the start site or point, the 10-35 bp sequence upstream of the start site, and the distance between the 10-35 bp sequences upstream of the start site. The start point is usually (90% of the time) a purine. Upstream of the start site is a 6 bp region that is recognizable in most promoters. The distance varies from 9-18 bp upstream of the start site, however, the consensus sequence is TATAAT. Another conserved hexamer is centered at 35 bp upstream of the start site. This consensus sequence is TTGACA. Additional promoter elements regulate the frequency of transcriptional initiation. The spacing between promoter elements frequently is flexible, so that promoter function is preserved when elements are inverted or moved relative to one another.

In certain embodiments, viral promoters may be used. These promoters may be extremely primitive or complex depending upon the virus. For example, some viral promoters like the T4 phage promoter may only contain an AT-rich sequence at 10 bp upstream of the start site, but not a consensus sequence 35 bp upstream of the start site.

In certain embodiments, the lac promoter, T7 promoter, T3, SP6, or tac promoter can be used to obtain high-level expression of the coding sequence of interest. The use of other bacterial, viral or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Also contemplated is the use of the native promoter to drive the expression of the nucleic acid sequence. Further, selection of a promoter that is regulated in response to specific physiologic signals can permit inducible expression of the gene product, e.g. heat shock promoters.

(ii) Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et. al., 1988 and Ausubel et. al., 1994, both incorporated herein by reference.

The term “expression vector” refers to a vector containing a nucleic acid sequence capable of being transcribed. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(iii) Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

(iv) Expression Systems

Numerous expression systems exist that comprise at least a part or all of the compositions discussed above. Prokaryote-based systems can be employed for use with the present invention to produce nucleic acid sequences, or their cognate polypeptides, proteins and peptides. Many such systems are commercially and widely available.

One skilled in the art is aware of the various prokaryote-based expression systems. Exemplary systems from PROMEGA include, but are not limited to, pGEMEX®-1 vector, pGEMX®-2 Vector, and Pinpoint control Vectors. Examples from STRATAGENE® include, but are not limited to, pBK Phagemid Vector, which is inducible by IPTG, pSPUTK In vitro Translation Vector, pET Expression systems, Epicurian Coli® BL21 Competent Cells and pDual™ Expression System.

III. Variants of β-lactamase Inhibitor Peptides

Amino acid sequence variants of the β-lactamase inhibitor peptide, can be substitutional, insertional or deletion variants. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

In certain embodiments of the invention, the peptide comprises X′₁X′₂X₁X₂X₃X₄ (SEQ ID NO:1); wherein X′₁ and X′₂ are arginine or lysine and are the same or different. It is also contemplated that X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine; and any of X₁, X₂, X₃, and X₄ may be the same or different. One with skill in the art is able to easily substitute amino acids, or design synthetic peptides, in order to determine which is the most appropriate peptide for binding to and inhibiting the target molecule. It is contemplated that the target molecule is a β-lactamase, and may be a variety of β-lactamases. One with skill in the art may determine which particular peptides bind with increased efficiency to the target. One such method is the SPOT synthesis array method, in which single amino acid substitutions are made from a starting peptide, and binding of the substituted peptides to a target molecule may be tested.

As used herein, “any of X₁, X₂, X₃, and X₄ may be the same or different” means that X₁, X₂, X₃, and X₄ may be substituted independently of each other with any of the group of non-negatively charged amino acids. For example, X₁may be the same as X₂, X₃, and X₄. They may all be histidine. Alternatively, X₁ may be glycine while X₂, X₃, and X₄ are all histidine. It is contemplated that X₁, X₂, X₃, and X₄ may all be different from each other. For example, X₁ may be glycine, X₂ may be arginine, X₃ may be histidine, and X₄ may be tyrosine. One with skill in the art realizes that such substitutions create a finite number of variations easily tested for the ability to bind β-lactamases by one with skill in the art in large scale parallel testing methods.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

IV. Beta-lactamases

Peptide inhibitors of β-lactamases of the present invention may be broad spectrum inhibitors. It is contemplated that β-lactamases that may be inhibited by the peptide inhibitors include class A and class C β-lactamases, although the present invention is not limited thereto. Examples of some β-lactamases that are contemplated are beta-lactamase TEM-1 [Serratia marcescens] GenBank Accession No. BAC81970 (SEQ ID NO:34); penicillinase TEM-1 [Pseudomonas aeruginosa] GenBank Accession No. CAA38430 (SEQ ID NO:35); beta-lactamase TEM-1 [Klebsiella pneumoniae] GenBank Accession No. AAP43782 (SEQ ID NO:36); Bla-1 [Staphylococcus aureus] GenBank Accession No. NP_{—}878025 (SEQ ID NO:37); Bla-1 beta-lactamase I [Bacillus anthracis] GenBank Accession No. AAK53749 (SEQ ID NO:38); and P99 [Enterobacter cloacae] GenBank Accession No. P05364 (SEQ ID NO:39).

One with skill in the art realizes that although some β-lactamases only share 30% homology, conserved sequences in the active site allow a single peptide inhibitor of β-lactamase to inhibit a broad spectrum of β-lactamases.

V. Proteinaceous Compositions

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 2
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one β-lactamase inhibitor peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The coding regions for these known genes may be amplified and/or expressed using the techniques disclosed herein or as would be know to those of ordinary skill in the art. Alternatively, various commercial preparations of proteins, polypeptides and peptides are known to those of skill in the art.

VI. Synthetic Peptides

The present invention also includes smaller β-lactamase inhibitor peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et. al, (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

VII. Pharmaceutical Compositions

Pharmaceutical compositions of the present invention comprise an effective amount of one or more β-lactamase inhibitor peptides or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one β-lactamase inhibitor peptides or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The β-lactamase inhibitor peptide may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The β-lactamase inhibitor peptide may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the β-lactamase inhibitor peptide is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

One with skill in the art realizes that the present invention relates to the novel finding that a six-mer peptide is sufficient to inhibit a broad spectrum of β-lactamases. This six-mer is a peptide wherein the first two amino acids may be arginine or lysine or some combination of the two, and the last four amino acids may be any combination of the non-negatively charged amino acids. These limitations cause there to be a finite number of variants of this six-mer. One with skill in the art is able to easily synthesize and test these peptides for binding to a peptide of interest, specifically a β-lactamase. One such method known to one with skill in the art is the SPOT synthesis method. See Frank, J Immunol Methods. Sep. 1, 2000;267(1):13-26, hereby incorporated by reference herein, for a review of the SPOT synthesis technique. Briefly, the method allows rapid and highly parallel synthesis of very large numbers of peptides. Further advantages are related to the easy adaptability to a wide range of assay and screening methods such as binding, enzymatic and cellular assays, which allow in situ screening of chemical libraries due to the special properties of the membrane supports.

SPOT synthesis is a solid phase synthesis concept in which SPOTs are defined by the depositing of small drops of reagent onto a predefined array of reaction sites on a coherent continuous solid phase supporting material, which functions as the polymeric solid phase supporting material; these SPOTs represent microreactors, in which solid phase syntheses can occur, if solvents with low vapor pressure are used. For example, peptide synthesis is performed by automated instrumentation and individual coupling reactions are monitored via fluorescence absorption of the Fmoc protecting group. Peptides are N-terminally acetylated and then side chain deprotected. The membrane bound peptides may be then screened for a functional characteristic, such a binding to a particular peptide. In this manner, one with skill in the art is able to screen a large number of peptides with ease. Specifically, large numbers of variants of the six-mer peptide inhibitor of β-lactamase may be screened. After parameters of β-lactamase binding are established, suitable peptides may be tested for ability to prevent the cleaving of cephalosporin, or other b-lactam antibiotics, by β-lactamase by methods described herein and known to those with skill in the art.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Phage Display

The Ph.D.-C7C (New England Biolabs, Inc.) was purchased and used to identify peptides that mediated binding to immobilized TEM-1 β-lactamase. The Ph.D.-C7C library consists of random sequence 7-mers fused to a minor coat protein (pIII) of M13 phage. Biopanning was performed by coating a micro-plate well with 200 μl of purified TEM-1 β-lactamase at a concentration of 40 μg/ml in 0.1 M NaHCO3 (pH8.6) at 4° C. overnight. The wells were then blocked with 200 μl of 5 mg/ml BSA in 0.1 M NaHCO₃ (pH 8.6), 0.02% NaN₃. After blocking, the C7C phage were input at 2×10¹¹ pfu/well in 200 μl wash buffer (1× TBS +0.1% (v/v) Tween-20) and incubated at room temperature for 1 hour. The wells were then washed ten times with 200 μl of wash buffer to remove unbound phages and bound phages were eluted by the addition of 200 μl of 0.2 M Glycine-HCl (pH 2.2) for 10 minutes at room temperature. The solution containing the eluted phages was neutralized by the addition of 25 μl of 1 M Tris pH 8.0.

The eluted phages were amplified by adding 150 μl of the neutralized solution to 25 ml of E. coli ER2738 cells [F′ proA+B+lacI_{q}Δ(lacZ)M15 zzf::Tn10(TetR)/fhuA2 gin V Δ(lac-proAB) thi-1 Δ(hsdS-mcrB)5] that had been grown to an OD₆₀₀ of 0.2 and incubating the culture at 37° C. for 4.5 hours. The phages were precipitated by the addition of ⅕ volume of 20% PEG/2.5 M NaCl and harvested by centrifugation. The titer of the resultant phage stock was determined by infecting E. coli ER 2738 cells with serial dilutions of the stock and counting the number of resultant plaques. The second and third rounds of biopanning were performed using the amplified phage stocks from the previous round. The panning was identical except the wash buffer for the second and third rounds was 1×TBS+0.5% Tween-20.

DNA sequencing was performed to determine if the library was converging on a particular sequence. For this purpose, 20 single plaques were selected after the first round of panning and 40 plaques were selected following the third round of panning and single stranded DNA was isolated and used as template for dideoxy DNA sequencing using the −96 sequencing primer (5′-CCCTCATAGTTAGCGTAACG-3′ (SEQ ID NO:12). The DNA sequencing reactions were performed with the ABI Big Dye Terminator Kit and the sequences were resolved using an ABI 3100 automated DNA sequencer.

### Example 2

### Phage ELISA

Phage stocks for ELISA experiments were prepared by adding 5 μl of phage supernatant from the clone of interest to a 25 ml culture of E. coli ER2738 that had been grown to an OD₆₀₀ of 0.1. The infected culture was then grown 4.5 hours at 37° C. The phages were harvested, precipitated and the titer was determined as described above. Wells of a micro-plate were coated with 20 μg/ml TEM-1 β-lactamase in 0.1 M NaHCO₃, pH 8.6 with 200 μl per well at 4° C. overnight and blocked with 200 μl blocking buffer at room temperature for 1 hour. Serial dilutions of the phage stock were performed into wash buffer (1× TBS, 0.5% Tween-20) and 200 μl of each dilution was added to the coated wells. The wells were then washed 6 times with 200 μl of wash buffer. Phages that bound β-lactamase and were retained in the well were detected with an anti-M13 phage antibody conjugated to horseradish peroxidase (HRP) (Amersham). HRP was detected after the addition of the ABTS indicator reagent by monitoring absorbance at 405 nm.

Synthesis and Screening of Cellulose-bound Peptides-Cellulose-bound peptides were prepared by automated SPOT synthesis (Auto-Spot Robot ASP 222, Intavis AG) as has been described in detail previously (Kramer et al., 1999; Kramer et al., 1993; Wenschuh et al., 2000). Amino-functionalized membranes were purchased from Intavis AG. The membranes are derivatized with a polyethylene glycol spacer with a length of 8 to 10 ethylene glycol units (Intavis AG). The spacer also contains a free amino group to initiate peptide synthesis. Peptide synthesis was performed with Fmoc protected amino acids as described previously (Wenschuh et al., 2000).

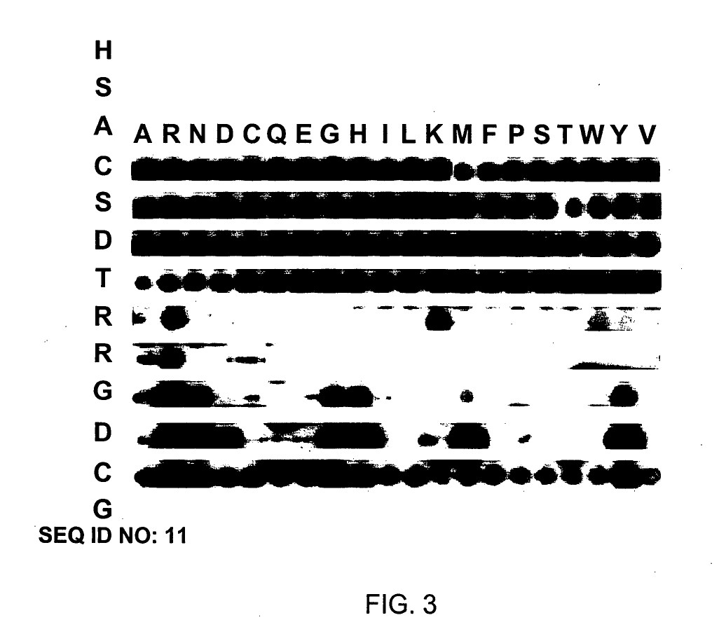

Two methods were used for detection of β-lactamase binding to the SPOT membranes. The first method involved detection of bound TEM-1 β-lactamase using polyclonal anti-β-lactamase sera (data in FIG. 3). For this experiment, the membrane was blocked overnight at 4° C. with SuperBlock Buffer (Pierce) that was supplemented with 1 mg/ml bovine serum albumin (BSA) and 1 mg/ml casamino acids. The membrane was washed with 1× TBS containing 1 mg/ml BSA and 1 mg/ml casamino acids (wash buffer) and TEM-1 β-lactamase was incubated with the membrane at 0.1 μg/ml in wash buffer at room temperature for 1 hour. The membranes were washed 4 times with wash buffer for 10 minutes each and a 1:10000 dilution of rabbit anti-β-lactamase serum in wash buffer was incubated with the membrane for 1 hour at room temperature. Detection was performed using a donkey anti-rabbit IgG conjugated to horseradish peroxidase (HRP). Bound antibody was detected using Amersham ECL chemiluminescent substrate and X-ray film.

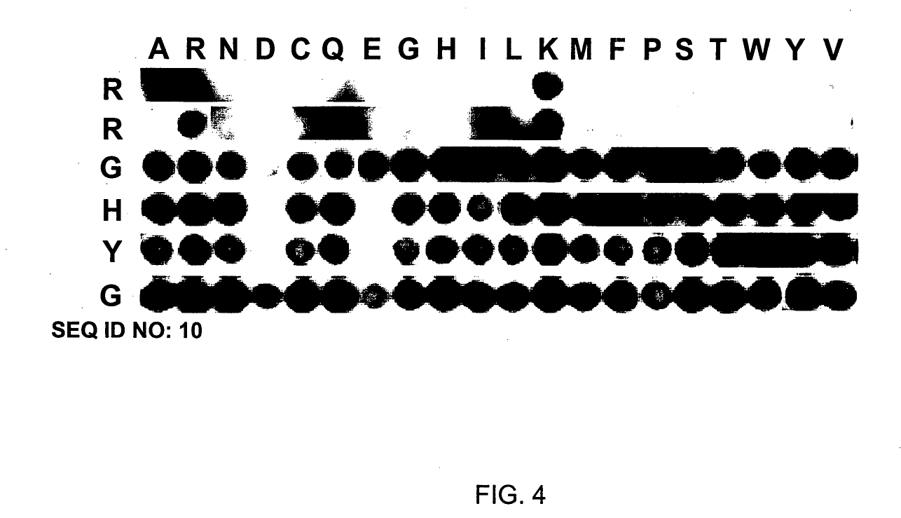

For the second method, membranes were blocked overnight at 4° C. with 1× TBS containing 1 mg/ml BSA and 1 mg/ml casamino acids. After washing with 1× TBS+0.5% Tween-20, the membranes were incubated with 0.9 μg/ml TEM-1 β-lactamase conjugated to HRP for 2 hours at room temperature. The membranes were then washed three times with 1× TBS-Tween-20 and bound β-lactamase-HRP was detected by chemiluminescence using X-ray film (ECL, Amersham) (data in FIG. 4). Quantitation of the intensity of spots was performed by densitometry using a VersaDoc Imaging System (BioRad, Inc.).

### Example 3

### Soluble Peptide Synthesis

All soluble peptides with the exception of the protein kinase substrates were prepared by solid-phase peptide synthesis using Fmoc protected monomers at the Baylor College of Medicine protein chemistry core facility using an ABI 433A synthesizer. The HSACSDTRRGDCG-NH₂ (SEQ ID NO:11) peptide was cyclized by the dropwise addition of ammonium hydroxide to the solution to pH 8.0. The progress of the reaction was monitored by reverse-phase high-pressure liquid chromatography (HPLC) and the final product was purified to >90% homogeneity by reverse phase HPLC. The HSAYSDTRRGDYG—NH₂ (SEQ ID NO:7), RRGHYY—NH₂ (SEQ ID NO:2) and AAGHYY—NH₂ (SEQ ID NO: 13) peptides were purified to >95% homogeneity by reverse phase HPLC. The identity of all synthesized peptides was verified by electrospray mass spectrometry at the Baylor College of Medicine protein chemistry core facility. The protein kinase substrates LRRASLG—NH₂ (SEQ ID NO: 14) and RRKASGP (SEQ ID NO: 15) were purchased from American Peptide Company, Inc. These peptides were purified to >99% and were analyzed by mass spectrometry by American Peptide Company.

### Example 4

### Purification of β-lactamase Proteins

The TEM-1 β-lactamase was purified to >90% homogeneity using a zinc chelating sepharose (fast flow) column (Pharmacia) and Sephadex G-75 gel filtration chromatography as previously described (Cantu III et al., 1996). The P99, Bla1 and IMP-1 enzymes were expressed in E. coli and purified as described previously (Materon and Palzkill, 2001; Materon et al., 2003; Zhang et al., 2001).

### Example 5

### β-lactamase Inhibition Assays

Inhibition assays were performed as described previously (Petrosino et al., 1999; Rudgers and Palzkill, 2001). Briefly, various concentrations of peptide were incubated with TEM-1 (45 nM), Bla1 (0.9 nM), P99 (0.6 nM) or IMP-1 (0.8 nM) β-lactamase for 1 hour in 50 mM phosphate buffer (pH 7.0) containing 1 mg/ml BSA. Following the incubation, the β-lactam substrate cephalosporin C (TEM-1, P99) or phenoxymethylpenicillin (Bla1) or nitrocefin (IMP-1) was added at a concentration at least 10-fold lower than the Kₘ of the substrate for the β-lactamase being tested. Hydrolysis of cephalosporin C was determined spectrophotometrically by measuring the decrease in A₂₈₀ (Δε=2390 M₋₁cm₋₁). Hydrolysis of phenoxymethylpenicillin (Pen V) by Bla1 was monitored by measuring the decrease in A₂₄₀ (Δε=570 M₋₁cm₋₁). Hydrolysis of nitrocefin was monitored by measuring the increase in A₄₉₅ (Δε=15,000 M₋₁cm₋₁). The concentration of peptide that reduced enzyme velocity by half was the IC₅₀ value. The Kᵢ was calculated from the IC₅₀ using the method of Cheng and Prusoff (Cheng and Prusoff, 1973) using the equation Kᵢ=IC₅₀/(1+[substrate]/K_{M}) where [substrate] is the concentration of β-lactam reporter substrate and K_{M} is the K_{M} value for the reporter for the enzyme being tested (Cheng and Prusoff, 1973). The data were fit to determine the IC₅₀ value using GraphPad Prism software.

The mode of inhibition of the RRGHYY—NH₂peptide (SEQ ID NO:2) was determined using a Beckman DU-640 spectrophotometer in 50 mM potassium phosphate (pH 7) containing 1 mg/mL BSA in a final volume of 0.5 mL. All assays were performed in triplicate. Hydrolysis of cephalosporin C by TEM-1 and P99 and hydrolysis of Pen V by Bla1 was determined spectrophotometrically at 280 nm and 240 nm, respectively, using the molar absorbances listed above. The peptide was allowed to incubate with TEM-1, P99, or Bla1 for one hour prior to the addition of cephalosporin C (TEM-1, P99) or Pen V (Bla1 ). TEM-1 (45 nM) and P99 (5 nM) assays were carried out at 0, 50, and 100 μM RRGHYY—NH₂ (SEQ ID NO:2) and increasing concentrations of cephalosporin C. Bla1 (0.9 nM) assays were carried out at 0, 15, 30 μm RRGHYY—NH₂ (SEQ ID NO:2) and increasing concentrations of Pen V. Kₘ and Vₘₐₓ values were determined from double reciprocal plots at each concentration of RRGHYY—NH₂ (SEQ ID NO:2).

### Example 6

### Selection of β-lactamase-Binding Peptides

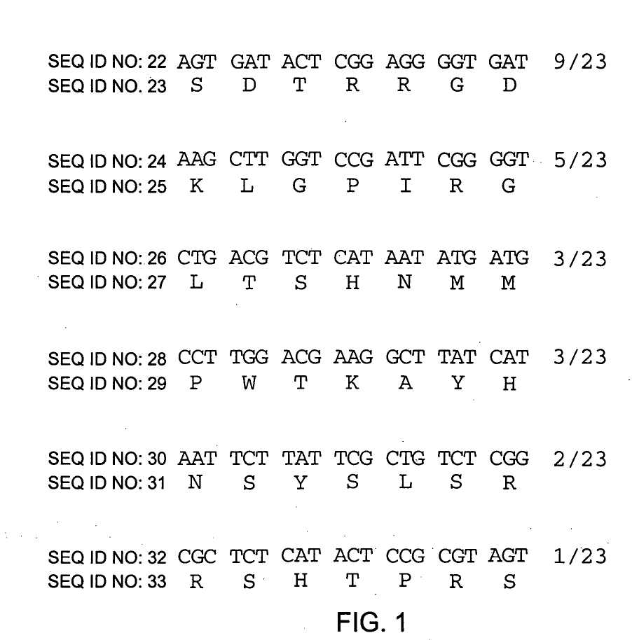

The goal of these experiments was to identify peptides from a random sequence library that would bind to the active site of TEM-1 β-lactamase. For this purpose, a phage display library displaying randomized 7-mer peptides flanked by a pair of cysteine residues was enriched for phage particles that bind to immobilized TEM-1 β-lactamase. After extensive washing to remove unbound phage, the bound phage were eluted from the immobilized β-lactamase and used to infect E. coli. The phage were amplified in the infected bacteria and used for another round of binding enrichment. After each round of binding enrichment, representative clones were randomly selected for DNA sequencing to determine if the library was converging on a particular sequence. After three rounds of panning, phages displaying six different peptide sequences were discovered (FIG. 1). Among these, phages displaying the sequence CSDTRRGDC (SEQ ID NO:16) were predominant, suggesting that this is the optimal peptide ligand for β-lactamase binding.

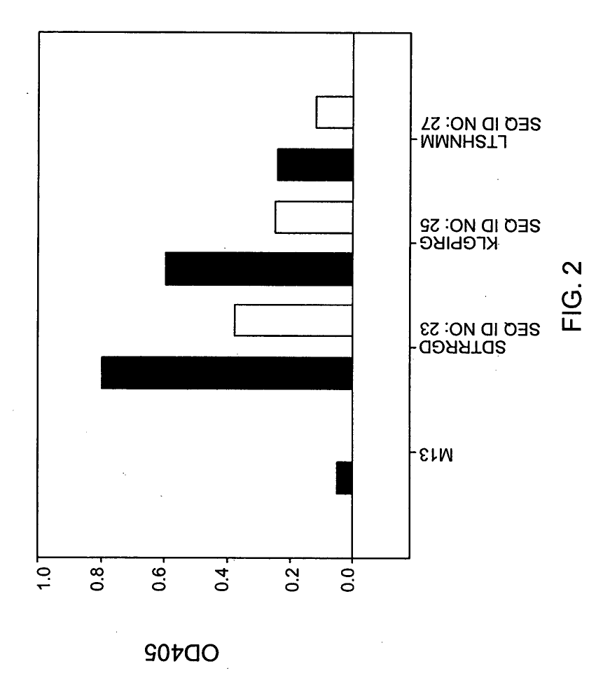

Phage ELISA was used to verify that the CSDTRRGDC (SEQ ID NO:16) peptide identified by the phage display experiments bound to immobilized TEM-1 β-lactamase (Huang et al., 1998)(FIG. 2). For these experiments, 10″ phage displaying the CSDTRRGDC (SEQ ID NO:16) peptide were added to a microtiter well that had been coated with 20 μg/ml TEM-1 β-lactamase.

After extensive washing, bound phages were detected with an anti-M13 phage antibody. Phage displaying two additional sequences, CKLGPIRGC (SEQ ID NO:17) and CLTSHNMMC (SEQ ID NO: 18), were also assayed for binding (FIG. 1). As a negative control, the phages were also tested for binding to the E. coli maltose binding protein (MBP). The results are consistent with the DNA sequencing results in that the CSDTRRGDC (SEQ ID NO:16) peptide exhibited the strongest binding to β-lactamase (FIG. 2). Phage displaying each of the peptides gave ELISA signals higher than background binding to MBP suggesting that all three peptides bind specifically to TEM-1 β-lactamase.

Although the CSDTRRGDC (SEQ ID NO:16) peptide was selected based on the ability to bind TEM-1 β-lactamase, it is possible that the peptide could bind without inhibiting function. To establish if the peptide inhibits TEM-1 β-lactamase, the peptide HSACSDTRRGDCG-NH₂ (SEQ ID NO: 11), which contains the 7-mer as well as flanking sequences from the phage, was synthesized, oxidized, and the disulfide-bonded version was purified. This peptide was used in a β-lactamase inhibition assay (Rudgers and Palzkill, 2001) and found to inhibit TEM-1 β-lactamase very weakly with a Kᵢ of approximately 3.5 mM. Therefore, although the peptide bound and inhibited the enzyme, it clearly needed further optimization to be a viable inhibitor or lead peptide.

### Example 7

### SPOT Synthesis

The SPOT synthesis method can be used to synthesize large arrays of synthetic peptides on planar cellulose supports (Frank, 1992; Reineke et al., 2001). This technique was used to synthesize an array of peptides containing all possible single amino acid substitutions for the 7-mer sequence and the flanking cysteines in the HSACSDTRRGDCG (SEQ ID NO:11) peptide. The array was screened for peptides that bound TEM-1 β-lactamase by incubating the filter with purified, soluble TEM-1 β-lactamase. After extensive washing, β-lactamase that was retained on the filter was detected with an anti-β-lactamase polyclonal antibody in a format similar to Western blotting (FIG. 3). The results indicated that the SDT region of the CSDTRRGDC (SEQ ID NO: 16) sequence did not contribute to binding since all substitutions function equally well at these positions. In contrast, the arginine residues appear to be critical for binding of the peptide to β-lactamase since substitutions at these positions eliminate binding (FIG. 3). In addition, the disulfide bond, which constrains the peptide, does not appear important for binding. In fact, a substitution of the C-terminal cysteine with tyrosine appears to increase binding strength of the peptide to β-lactamase (FIG. 3). Similarly, certain substitutions at the C-terminal glycine and aspartate positions of the CSDTRRGDC (SEQ ID NO: 16) result in increased binding to β-lactamase.

In order to test whether substitution of the cysteine residues increases binding affinity as suggested by the array results, a soluble peptide was synthesized with the cysteines replaced by tyrosine residues. The HSAYSDTRRGDYG-NH₂ (SEQ ID NO:7) peptide was found to inhibit TEM-1 β-lactamase with a Kᵢ of 298 μm (Table 1). Therefore, replacement of the cysteines residues with tyrosines results in an approximately 10-fold increase in binding affinity, which is consistent with the qualitative result from the SPOT array.

Peptide binding to β-lactamase was optimized further by synthesis of another SPOT array. The previous array suggested that only the RRGDYG (SEQ ID NO:8) region of the peptide contributed to β-lactamase binding. In addition, the results of the previous array suggested that substitution of the aspartate residue (RRGDYG SEQ ID NO:8) with histidine to give a peptide with the sequence RRGHYG (SEQ ID NO: 10) would result in improved binding. Therefore, the RRGHYG (SEQ ID NO:10) peptide and all single amino acid substitutions of this peptide were synthesized on the SPOT array (FIG. 4). To ensure that the histidine substitution does in fact result in tighter binding, the RRGDYG (SEQ ID NO:8) peptide was also synthesized on the array for comparison. The array was probed with purified TEM-1 β-lactamase that had been conjugated to horseradish peroxidase (HRP), which allowed for direct detection of binding upon addition of the chemiluminescent HRP substrate. As predicted based on the results of the previous array, the RRGHYG (SEQ ID NO: 10) peptide gave a stronger binding signal than the RRGDYG (SEQ ID NO:8) control peptide (FIG. 4). The results also indicated that the two N-terminal arginine residues are important for binding to β-lactamase in that only arginine or lysine residues can substitute at these positions. In contrast, the glycine residue at position three (RRGHYG, SEQ ID NO: 10) can be substituted by all but negatively charged amino acids. Similarly, the histidine residue at position four (RRGHYG) (SEQ ID NO: 10) can also be substituted by several different amino acids but not by negatively charged residues. Several different residues can substitute for the tyrosine at position five (RRGHYG) (SEQ ID NO: 10) although many of these substitutions, particularly negatively charged residues, result in less effective binding than the peptide containing tyrosine at this position. Finally, several amino acids, including tyrosine, when substituted for glycine at position six (RRGHYG) (SEQ ID NO: 10) result in a more intense spot and presumably tighter binding. In summary, the SPOT synthesis results indicate a six-mer peptide can bind to TEM-1 β-lactamase and that the N-terminal arginine residues are critical for this binding.

The soluble RRGHYY—NH₂ (SEQ ID NO:2) peptide was synthesized and tested for inhibition of TEM-1 β-lactamase to validate the SPOT synthesis results. This peptide includes the substitution of tyrosine for glycine at position six, which appears to contribute to tighter binding based on the array results (FIG. 4). The peptide was found to inhibit TEM-1 β-lactamase with a Kᵢ of 136 μM, which is an approximately 2-fold improvement in affinity relative to the HSAYSDTRRGDYG-NH₂ (SEQ ID NO:7) peptide, and a 25-fold improvement relative to the disulfide bonded HSACSDTRRGDCG-NH₂ (SEQ ID NO: 11) peptide originally discovered by phage display (Table I).

TABLE 3

Ki determinations for inhibition of
β-lactamases by peptides.

Ki (□M)

| | | | |
|---|---|---|---|
| Peptide | TEM-1 | B. anthracis Bla1 | E. cloacae P99 |

| | | | |
|---|---|---|---|
| HSAYSDTRRGDYG-NH₂ | 298 ± 36 | 70 ± 8 | 254 ± 30 |
| (SEQ ID NO:7) | | | |
| RRGHYY-NH₂ | 136 ± 20 | 42 ± 7 | 140 ± 13 |
| (SEQ ID NO:2) | | | |
| AAGHYY-NH₂ | 438 ± 30 | 72 ± 6 | 468 ± 9 |
| (SEQ ID NO:13) | | | |
| RR-NH₂ | >2000^{a} | >2000 | >2000 |
| (SEQ ID NO:19) | | | |

^{a}No inhibitory activity detected for this peptide against this enzyme up to 2 mM peptide concentration.

The SPOT synthesis results suggest the arginine residues within the RRGHYY (SEQ ID NO:2) peptide play an important role in β-lactamase binding. This result was confirmed by determining the inhibition constant for the soluble peptide AAGHYY—NH₂ (SEQ ID NO: 13). Substitution of the arginines with alanine residues resulted in a 3-fold increase in Kᵢ for inhibition of TEM-1 β-lactamase indicating they contribute to inhibition (Table I).

Because the arginine residues are important for binding, the ability of soluble Larginine to inhibit TEM-1 β-lactamase was assayed. Soluble L-arginine at concentrations up to 2.5 mM had no effect on β-lactamase activity, indicating that L-arginine is not an inhibitor of the enzyme. In order to address the possibility that the dipeptide Arg-Arg could inhibit the enzyme, an Arg-Arg-NH₂ peptide was tested and found to have no effect on β-lactamase activity at concentrations up to 2.0 mM. Therefore, although the Arg-Arg region of the peptide appears important for binding β-lactamase, it is not sufficient. It is possible, however, that the presence of the Arg-Arg motif in the context of a peptide of similar size to RRGHYY—NH₂ (SEQ ID NO:2) is sufficient for inhibiting β-lactamase. This possibility was tested using the commercially available protein kinase substrates LRRASLG-NH₂ (SEQ ID NO:20) and RRKASGP (SEQ ID NO:21) (Kemp et al., 1977; Pomerantz et al., 1977). It was found that these peptides had no effect on TEM-1 β-lactamase activity at concentrations up to 4 mM. Therefore, the specific sequence of the RRGHYY—NH₂ (SEQ ID NO:2) peptide appears important for inhibition.

### Example 8

### Inhibition of other β-lactamases

Although the peptides were selected and optimized for binding to the TEM-1 β-lactamase of gram-negative bacteria, it is possible these peptides may inhibit other β-lactamases as well. Recently, the gene encoding the Bla1 class A β-lactamase from Bacillus anthracis was cloned and the enzyme was expressed and purified from E. coli (Chen et al., 2003; Materon et al., 2003). The HSAYSDTRRGDYG-NH₂ (SEQ ID NO:7) and RRGHYY—NH₂ (SEQ ID NO:2) peptides were tested for inhibition of the B. anthracis Bla1 enzyme (Table 1). Surprisingly, despite having been optimized to bind TEM-1 β-lactamase, each of the peptides was a more effective inhibitor of the Bla1 enzyme than the TEM-1 enzyme. For example, the RRGHYY—NH₂ (SEQ ID NO:2) peptide inhibits Bla1 with a Kᵢ of 42 μM, which is approximately 3-fold lower than the Kᵢ for inhibition of TEM-1 β-lactamase. Similarly, the HSAYSDTRRGDYG-NH₂ (SEQ ID NO:7) peptide inhibits Bla1 with a Kᵢ of 70 μm, which is approximately 4-fold lower than the Kᵢ for inhibition of TEM-1 β-lactamase. A possible explanation for the broad inhibition profile of these peptides is that the catalytic residues in the active site pocket of class A enzymes such as TEM-1 and Bla1 are highly conserved (Frere et al., 1999). Thus, although the enzymes are only about 30% identical, the active site pockets are very similar.

The peptides were also tested for inhibition of the class C β-lactamase, P99, from the gram-negative bacterium Enterobacter cloacae (Dubus et al., 1996; Lobkovsky et al., 1993). The class A and class C enzymes have a similar fold and contain conserved amino acids that act similarly in catalysis (Lobkovsky et al., 1993). However, there are also many differences in the active site, which may explain why the mechanism based inhibitors sulbactam and clavulanic acid are poor inhibitors of class C enzymes (Bush, 2002). Although the HSACSDTRRGDCG-NH₂ (SEQ ID NO: 11)peptide did not detectably inhibit P99 at concentrations up to 800 μM, the HSAYSDTRRGDYG-NH₂ (SEQ ID NO:7) peptide inhibited P99 with a Kᵢ of 254 μM and the RRGHYY—NH₂ (SEQ ID NO:2) peptide inhibited the enzyme with a Kᵢ of 140 μm. Thus, the RRGHYY—NH₂ (SEQ ID NO:2) peptide inhibits class A and class C enzymes with a similar efficiency.

Because the RRGHYY—NH₂ (SEQ ID NO:2) peptide inhibited several enzymes, the specificity of these interactions was investigated further. The replacement of the arginines with alanines in the AAGHYY—NH₂ (SEQ ID NO: 13) peptide resulted in a 3-fold increase in Kᵢ for the P99 enzyme and a two-fold increase for Bla1 indicating the arginine residues are important for binding both of these enzymes (Table 1). In addition, it was found that, similar to the results with TEM-1, the dipeptide Arg-Arg-NH2 had no effect on the β-lactamase activity of these enzymes at concentrations up to 2.0 mM (Table 1). These results suggest that the sequence requirements of the peptide for inhibition of the β-lactamases are similar. Because the arginine residues are important for inhibition, it is possible that a charge interaction contributes to binding. However, there is not a correlation between binding and the pI of the enzyme in that the pI of TEM-1 is 5.4 while that of Bla1 and P99 are 9.1 and 8.7, respectively.

Finally, it was found that the RRGHYY—NH₂ (SEQ ID NO:2) peptide displayed no inhibition of the class B zinc-metallo-β-lactamase IMP-1 at concentrations up to 400 μm. This result is not surprising in that the zinc metallo-enzymes have a completely different fold and utilize zinc ions rather than a catalytic serine (Wang et al., 1999). Taken together, these results suggest the RRGHYY—NH₂ (SEQ ID NO:2) peptide is a broad-spectrum inhibitor of active-site serine β-lactamases and that this inhibition is dependent on the specific sequence of the peptide.

### Example 9

### Mode of RRGHYY—NH₂ Inhibition

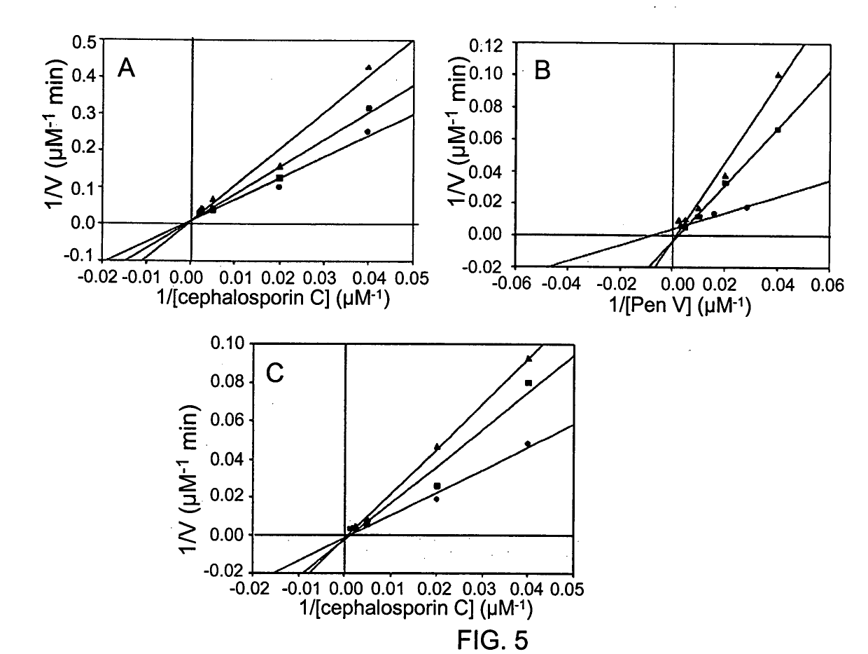

In order to gain further insight into the mechanism by which the RRGHYY—NH₂ (SEQ ID NO:2) peptide inhibits β-lactamases, the inhibition patterns with respect to the TEM-1, Bla1 and P99 enzymes were determined. The RRGHYY—NH₂ (SEQ ID NO:2) peptide demonstrated a competitive inhibition pattern with respect to β-lactam substrates for each of the enzymes tested (FIG. 5). This inhibition pattern suggests that the peptide acts similarly for each target by binding at or near the active site of the β-lactamases to block entry of the β-lactam substrate.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

U.S. Patents and Patent Application Publications

U.S. Pat. No. 5,677,153

20010007754

20010006959

Non-Patent Publications

Bush, K. (1998) Clin. Infect. Dis., 27, S48-S53.
Bush, K. (2002) Curr. Opin. Invest. Drugs, 3, 1284-1290.
Cantu III, C., Huang, W. and Palzkill, T. (1996) J. Biol. Chem., 271, 22538-22545.
Carfi, A., Pares, S., Duee, E., Galleni, M., Duez, C., Frere, J. M. and Dideberg, O. (1995) EMBO J., 14, 4914-4921.
Cavallo, J. D., Ramisse, F., Girardet, M., Vaissaire, J., Mock, M. and Hernandez, E. (2002) Antimicrob. Agents Chemother., 46, 2307-2309.
Charnas, R. and Knowles, J. (1981) Biochemistry, 20, 2732-2737.
Chen, Y., Succi, J., Tenover, F. C. and Koehler, T. M. (2003) J. Bacteriol., 185, 823-830.
Cheng, Y. and Prusoff, W. H. (1973) Biochem. Pharmacol., 22, 3099-3108.
Clackson, T. and Wells, J. (1994) Trends Biotechnol., 12, 173-184.
Cochran, A. G. (2001) Curr. Opin. Chem. Biol., 5, 654-659.
Dostmann, W. R. G., Nickl, C., Thiel, S., Tsigelny, I., Frank, R. and Tegge, W. J. (1999) Pharmacol. Ther., 82, 373-387.
Dubus, A., Lendent, P., Lamotte-Brasseur, J. and Frere, J.-M. (1996) Proteins, 25, 473-485.
Frank, R. (1992) Tetrahedron, 48, 9217-9232.
Frere, J. M., Dubus, A., Galleni, M., Matagne, A. and Amicosante, G. (1999) Biochem. Soc. Trans., 27, 58-63.
Ghuysen, J.-M. (1991) Annu. Rev. Microbiol., 45, 37-67.
Henquell, C., Chanal, C., Sirot, D., Labia, R. and Sirot, J. (1995) Antimicrob. Agents Chemother., 39, 427-430.
Huang, W., Petrosino, J. and Palzkill, T. (1998) Antimicrob. Agents Chemother., 42, 2893-2897.
Imtiaz, U., Manavathu, E., Mobashery, S. and Lerner, S. (1994) Antimicrob. Agents Chemother., 38, 1134-1139.
Kemp, B. E., Graves, D. J., Benjamini, E. and Krebs, E. G. (1977) J Biol. Chem., 252, 4888-4894.
Knoblauch, N. T. M., Rudiger, S., Schonfeld, H. J., Driessen, A. J. M., Schneider-Mergener, J. and Bukau, B. (1999) J Biol. Chem., 274, 34219-34225.
Kramer, A., Keitel, T., Winkler, K., Stocklein, W., Hohne, W. and Schneider-Mergener, J. (1997) Cell, 91, 799-809.
Kramer, A., Reineke, U., Dong, L., Hoffman, B., Winkler, D., Volkmer-Engert, R. and Schneider-Mergener, J. (1999) J Peptide Res., 54, 319-327.
Kramer, A., Volkmer-Engert, R., Malin, R., Reineke, U. and Schneider-Mergener, J. (1993) Pept. Res., 6, 314-319.
Livermore, D. M. (1995) Clin. Microbiol. Rev., 8, 557-584.
Lobkovsky, E., Moews, P. C., Liu, H., Zhao, H., Frere, J.-M. and Knox, J. R. (1993) Proc. Natl. Acad. Sci. USA, 90, 11257-11261.
Majiduddin F K, Palzkill T. Genetics. February 2003;163(2):457-66.
Massova, I. and Mobashery, S. (1998) Antimicrob. Agents Chemother., 42, 1-17.
Materon, I. C. and Palzkill, T. (2001) Protein Sci., 10, 2556-2565.
Materon, I. C., Queenan, A. M., Koehler, T. M., Bush, K. and Palzkill, T. (2003) Antimicrob. Agents Chemother., 47, 2040-2042.
Nieto, M. and Perkins, H. R. (1971) Biochem. J., 123, 773-787.
Petrosino, J., Cantu III, C. and Palzkill, T. (1998) Trends Microbiol., 6, 323-327.
Petrosino, J., Rudgers, G., Gilbert, H. and Palzkill, T. (1999) J. Biol. Chem., 274, 2394-2400.
Pomerantz, A. H., Allfrey, V. G., Merrifield, R. B. and Johnson, E. M. (1977) Proc. Natl. Acad. Sci. USA, 74, 4261-4265.
Rasched, I. and Oberer, E. (1986) Microbiol. Rev., 50, 401-427.
Reimer, U., Reinke, U. and Schneider-Mergener, J. (2002) Curr. Opin. Biotechnol., 13, 315-320.
Reineke, U., Volkmer-Engert, R. and Schneider-Mergener, J. (2001) Curr. Opin. Biotechnol., 12, 59-64.
Rudgers, G. W., Huang, W. and Palzkill, T. (2001) Antimicrob. Agents Chemother., 45, 3279-3286.
Rudgers, G. W. and Palzkill, T. (2001) Protein eng. 14, 487-492.
Rudgers G W, Palzkill T.(2003) Methods Mol Biol.; 230:71-81.
Smith, G. P. (1985) Science, 228, 1315-1317.
Smith, G. P. and Petrenko, V. A. (1997) Chem. Rev., 97, 391-410.
Strynadka, N. C. J., Jensen, S. E., Alzari, P. M. and James, M. N. G. (1996) Nature Struct. Biol., 3, 290-297.
Strynadka, N. C. J., Jensen, S. E., Johns, K., Blanchard, H., Page, M., Matagne, A., Frere, J.-M. and James, M. N. G. (1994) Nature, 368, 657-660.
Wang, Z., Fast, W., Valentine, A. M. and Benkovic, S. J. (1999) Curr. Opin. Chem. Biol., 3, 614 622.
Wenschuh, H., Gausepohl, H., Germeroth, L., Ulbricht, M., Matuschewski, H., Kramer, A., Volkmer-Engert, R., Heine, N., Ast, T., Scham, D. and Schneider-Mergener, J. (2000) In Fenniri, H. (ed.) Combinatorial Chemistry: A Practical Approach. Oxford University Press, New York, pp. 95-116.
Wrighton, N. C., Farrell, F. X., Chang, R., Kashyap, A. K., Barbone, F. P., Mulcahy, L. S., Johnson, D. L., Barrett, R. W., Jolliffe, L. K. and Dower, W. J. (1996) Science 273, 458-463.
Zhang, Z., Yu, Y., Musser, J. M. and Palzkill, T. (2001) J Biol. Chem., 276, 46568-46574.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A peptide inhibitor of β-lactamase comprising X′₁X′₂X₁X₂X₃X₄ (SEQ ID NO:1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine, and wherein any of X₁, X₂, X₃, and X₄ are the same or different; and
wherein the peptide inhibitor of β-lactamase has an inhibition constant (Kᵢ) for the β-lactamase of no greater than about 500 μm.

2. The peptide inhibitor of claim 1, wherein the peptide inhibitor of β-lactamase comprises RRGHYY (SEQ ID NO:2).

3. The peptide inhibitor of claim 1, wherein the peptide inhibitor of β-lactamase comprises a peptide selected from the group consisting of RRX₁HYY (SEQ ID NO:3), RRGX₂YY (SEQ ID NO:4), RRGHX₃Y (SEQ ID NO:5), and RRGHYX₄ (SEQ ID NO:6).

4. The peptide inhibitor of claim 1, wherein the peptide inhibitor of β-lactamase comprises a peptide selected from the group consisting of HSAYSDTRRGDYG (SEQ ID NO:7), RRGDYG (SEQ ID NO:8), RRGDYH (SEQ ID NO:9), and RRGHYG (SEQ ID NO:10).

5. The peptide inhibitor of claim 1, wherein the β-lactamase is a class A or class C β-lactamase.

6. The peptide inhibitor of claim 5, wherein the β-lactamase is TEM-1, Bla1, or P99.

7. The peptide inhibitor of claim 1, wherein the inhibition constant (Kᵢ) is less than about 100 μm.

8. The peptide inhibitor of claim 1, further comprising a C-terminal —NH₂.

9. A pharmaceutical composition comprising a therapeutically effective amount of the peptide inhibitor of β-lactamase of claim 1.

10. The pharmaceutical composition of claim 8, wherein the peptide inhibitor of β-lactamase comprises RRGHYY (SEQ ID NO:2).

11. The pharmaceutical composition of claim 8, wherein the peptide inhibitor of β-lactamase comprises a peptide selected from the group consisting of RRX₁HYY (SEQ ID NO:3), RRGX₂YY (SEQ ID NO:4), RRGHX₃Y (SEQ ID NO:5), and RRGHYX₄ (SEQ ID NO:6).

12. The pharmaceutical composition of claim 8, wherein the peptide inhibitor of β-lactamase comprises a peptide selected from the group consisting of HSAYSDTRRGDYG (SEQ ID NO:7), RRGDYG (SEQ ID NO:8), RRGDYH (SEQ ID NO:9), and RRGHYG (SEQ ID NO:10).

13. The pharmaceutical composition of claim 8, further comprising a therapeutically effective amount of a β-lactam antibiotic.

14. The pharmaceutical composition of claim 12, wherein said β-lactam antibiotic is selected from the group consisting of penicillin, cephalosporin, monobactam and carbapenem antibiotics.

15. The pharmaceutical composition of claim 13, wherein said antibiotic is a penicillin.

16. The pharmaceutical composition of claim 14, wherein said penicillin is selected from the group consisting of azlocillin, methicillin, nafcillin, cloxacillin, dicloxacillin, oxacillin, ampicillin, bacampicillin, carbenicillin, ticarcillin, mezlocillin, penicillin, and piperacillin.

17. The pharmaceutical composition of claim 13, wherein said antibiotic is a cephalosporin.

18. The pharmaceutical composition of claim 16, wherein said cephalosporin is selected from the group consisting of cefoxitin, cefoperazone, ceftazidime, ceftriaxone, cefadroxil, cefazolin, cephalexin, cephaloridine, cephalothin, cephapirin, cephradine, cefaclor, cefamandole, cefonicid, ceforanide, cefprozil, cefuroxime, loracarbef, cefmetazole, cefotetan, cefixime, cefotaxime, cefpodoxime, and ceftizoxime.

19. A DNA expression vector comprising a promoter operatively linked to a nucleotide sequence
wherein the nucleotide sequence encodes a peptide inhibitor of β-lactamase comprising X′₁X′₂X₁X₂X₃X₄ (SEQ ID NO: 1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine and wherein any of X₁, X₂, X₃, and X₄ are the same or different; and
wherein the peptide inhibitor of β-lactamase has an inhibition constant (Kᵢ) for the β-lactamase of no greater than about 500 μm.

20. A host cell capable of expressing a peptide inhibitor of β-lactamase comprising X′₁X′₂X₁X₂X₃X₄ (SEQ ID NO:1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine and wherein any of X₁, X₂, X₃, and X₄ are the same or different; and
wherein the peptide inhibitor of β-lactamase has an inhibition constant (Kᵢ) for the β-lactamase of no greater than about 500 μm.

21. A method of inhibiting a β-lactamase comprising contacting the β-lactamase with a peptide inhibitor of β-lactamase, wherein the peptide inhibitor of β-lactamase comprises X′₁X′₂X₁X₂X₃X₄ (SEQ ID NO: 1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine and wherein any of X₁, X₂, X₃, and X₄ are the same or different; and
wherein the peptide inhibitor of β-lactamase has an inhibition constant (Kᵢ) for the β-lactamase of no greater than about 500 μm.

22. A method of inhibiting the growth of a microorganism comprising contacting the microorganism with a β-lactam antibiotic and a peptide inhibitor of β-lactamase, wherein the peptide inhibitor of β-lactamase comprises X′₁X′₂X₁X₂X₃X₄ (SEQ ID NO:1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine and wherein any of X₁, X₂, X₃, and X₄ are the same or different; and
wherein the peptide inhibitor of β-lactamase has an inhibition constant (Kᵢ) for the β-lactamase of no greater than about 500 μm.

23. The method of claim 21, wherein the microorganism is selected from the group consisting of Staphylococcus aureus, Staphylococcus epidermidis and other coagulase-negative staphylococci, Streptococcus pyogenes, Streptococcus pneumoniae, Streptococcus agalactiae, Enterococcus species, Corynebacterium dipahtheriae, Listeria monocytogenes, Bacillus anthracis, Neisseria meningitidis, Neisseria gonorrhoeae, Moraxella catarrhalis, Vibrio cholerae, Campylobacter jejuni, Enterobacteriaceae (includes: Escherichia, Salmonella, Klebsiella, Enterobacter), Pseudomonas aeruginosa, Acinetobacter species, Haemophilus influenzae, Clostridium tetani, Clostridium botulinum, Bacteroides species, Prevotella species, Porphyromonas species, Fusobacterium species, Mycobacterium tuberculosis, and Mycobacterium leprae.

24. The method of claim 21, wherein the microorganism is resistant to one or more β-lactam antibiotics.

25. A method of treating a subject infected with a microorganism comprising administering to the subject a therapeutically effective amount of a β-lactam antibiotic and a peptide inhibitor of β-lactamase, whererein the peptide inhibitor of β-lactamase comprises X′₁X′₂X₁X₂X₃X₄ (SEQ ID NO: 1); wherein X′₁ and X′₂ are arginine or lysine and are the same or different;
wherein X₁, X₂, X₃, and X₄ are selected from the group consisting of alanine, arginine, asparagine, cysteine, glutamine, glycine, histidine, isoleucine, leucine, lysine, methionine, phenylalanine, proline, serine, threonine, tryptophan, tyrosine, and valine and wherein any of X₁, X₂, X₃, and X₄ are the same or different; and
wherein the peptide inhibitor of β-lactamase has an inhibition constant (Kᵢ) for the β-lactamase of no greater than about 500 μm.
